(19) United States
(12) Patent Application Publication
(10) Pub. No.: US 2008/0138288 A1
(43) Pub. Date: Jun. 12, 2008

(54) COMBINATIONS FOR TREATMENT OF NEOVASCULATURE

(75) Inventors: Gregory M. LANZA, St. Louis, MO (US); Samuel A. Wickline, St. Louis, MO (US)

Correspondence Address: MORRISON & FOERSTER LLP, 12531 HIGH BLUFF DRIVE, SUITE 100, SAN DIEGO, CA, 92130-2040, US

(73) Assignee: Washington University, St. Louis, MO (US)

(21) Appl. No.: 11/865,603

(22) Filed: Oct. 1, 2007

Related U.S. Application Data
(60) Provisional application No. 60/848,131, filed on Sep. 29, 2006.
(60) Provisional application No. 60/861,650, filed on Nov. 28, 2006.

(51) Int. Cl. A61K 49/06; A61K 31/4745; A61K 31/336; A61K 31/436; A61P 9/10; A61P 35/00; A61P 19/02

(52) U.S. Cl. 424/9.2; 514/475; 514/291; 514/449

(57) ABSTRACT

Long-term treatment of conditions associated with neovasculature is effected by combining targeted acute antiangiogenic agent treatment with chronic treatment with an additional drug.

### CROSS-REFERENCE TO RELATED APPLICATIONS

This application claims priority from U.S. provisional application Ser. No. 60/848,131 filed 29 Sep. 2006 and from provisional application Ser. No. 60/861,650 filed 28 Nov. 2006. The contents of these documents are incorporated herein by reference in their entirety.

### STATEMENT OF RIGHTS TO INVENTION MADE UNDER FEDERALLY SPONSORED RESEARCH

This work was supported in part by grants from agencies of the U.S. government. The U.S. government has certain rights in this invention.

### TECHNICAL FIELD

The invention relates to treatment of conditions associated with unwanted neovasculature, e.g., atherosclerosis. More precisely, it concerns combining targeted acute antiangiogenic agents with chronic treatment using an additional agent.

### BACKGROUND ART

PCT publication WO 2005/077407 published 25 Aug. 2005 describes treatment of atherosclerosis using targeted carrier compositions comprising antiangiogenic agents in “acute” protocols as well as imaging affected vessels using targeted particles associated with MRI contrast agents. (Enhancement of MRI signaling is indicative of enhanced angiogenesis.) This has been shown, for example, by Winter, et al., Circulation (2003) 108:2270-2274, by Brooks, et al., Cell (1994) 79:1157-1164, and by Winter, P. M., et al., Arterioscler. Thromb. Vasc. Biol. (2006) 26:2103-2109. Thus, as taught by these documents, delivery to the vasa vasorum using nanoparticles of both antiangiogenic drugs and imaging agents, and subsequent imaging are known in the art.

One disadvantage of the use of antiangiogenesis agents only in an “acute” regimen to treat atherosclerotic plaques is that the regimen is, indeed, acute. Thus, once treatment stops, the angiogenesis can resume and the plaques re-form. The present invention solves this problem and addresses additional conditions associated with angiogenesis by administering antiangiogenesis or other stabilizing drugs in a “chronic” protocol in combination with acute treatment.

### DISCLOSURE OF THE INVENTION

It has been found that by administering a antiangiogenic or other beneficial drug on a chronic basis in combination with acute treatment, the regression of plaques resulting from atherosclerosis can be maintained, and other conditions associated with unwanted neovasculature can be stabilized or cured. Thus, in one aspect, the invention is directed to a method to inhibit angiogenesis, which method comprises administering to a subject in need of such treatment an effective amount of at least one acute antiangiogenic drug targeted to neovasculature in an acute regimen in combination with administering an antiangiogenic or other beneficial drug in a chronic regimen. The acute treatment is defined as short-term, while the chronic treatment is more prolonged. Any effective combination protocol involving acute/chronic treatments can be used.

In addition, the progress of treatment can be monitored using various imaging techniques, preferably MRI imaging, and preferably targeted MRI imaging.

### BRIEF DESCRIPTION OF THE DRAWINGS

FIG. 1 is a graph comparing the effect of various treatment protocols with control on angiogenesis associated with plaque formation.

FIG. 2 is a graph showing the effect of targeted fumagillin on angiogenesis associated with plaque formation as measured by MRI signal enhancement in the presence and absence of a cholesterol-rich diet.

FIGS. 3A and 3B are graphs showing the effect of administering fumagillin with and without chronic administration of statin on angiogenesis associated with plaque formation, again measured as MRI signal enhancement. FIG. 3A shows the effect of the acute and chronic treatments, each done alone, and FIG. 3B shows the effect of combining administration of fumagillin on an acute basis with administration of statin on a chronic basis.

### MODES FOR CARRYING OUT THE INVENTION

The invention resides in the concept that combining acute treatment of a condition associated with unwanted neovasculature with chronic treatment using an additional beneficial agent not only alleviates the negative effects of the condition and stabilizes or reverses it, but is able to maintain the effect of this stabilization or reversal over a long time period. In addition, the invention offers a convenient method to monitor the efficacy of treatment over any desired portion, or for longer than, the period of chronic treatment. Thus, by taking advantage of continuous monitoring, for example, using targeted MRI contrast agents according to the method of the invention, it is possible to determine whether additional acute administration of antiproliferative agents is desirable.

Acute treatments are generally either a single bolus or continuous administration, typically over several hours or possibly a few days, or repetitive such treatments of relatively short duration over a confined time period of hours, days or weeks. Specifically, “acute” treatment is defined as administering an antiangiogenic agent to a subject for a time period that is substantially less that the time period over which success of the treatment is measured. Typically, the treatment period is 10% or less, more preferably 5% or less, or 1% or less of a period of evaluation of the treatment over which it is intended that the effect of the treatment will be at least partially sustained. Thus, if the evaluation period is 20 days, the period of administration would be not more than two days or not more than one day or not more than one-half day. In many cases, acute administration is much shorter and comprises only 0.1% or 0.01% of the evaluation period. The evaluation period of, perhaps, six weeks, might involve an administration period of only several hours or less. As, in one embodiment of the invention, the acute administration can be by intravenous injection, typically, the time period over which an injection will take place will be of the order of several hours, wherein the time for evaluation would be one month or two months.

Chronic treatment, on the other hand, is more prolonged and typically would require at least a week of repetitive or continuous treatments and such treatments could continue over several weeks or months or even years. While not a requirement of the invention, it is preferred that the chronic administration be suitable for self-administration such as by an oral route or by an inhalant. Acute treatment may have these characteristics as well, but may also but not necessarily be administered in a care center or hospital setting.

Thus, “chronic treatment” refers to treatment that is maintained on a repetitive basis over at least one-half to three-fourths of the evaluation period and preferably all of the evaluation period. Typically, chronic administration is repetitive such as a once or twice per day administration or every day or every other day over a substantial portion of the evaluation period.

The treatments associated with the present invention are designed to inhibit the formation of new blood vessels and/or to diminish the level of neovasculature already present. Thus, the methods of the invention are appropriate to conditions where angiogenesis is a problem. Such conditions include atherosclerosis and in particular atherosclerotic plaques, tumors, in particular those tumors that are characterized by particularly troublesome angioproliferation, such as Kaposi's sarcoma, arthritis (including rheumatoid arthritis and osteoarthritis), the proliferative retinopathies, such as that associated with diabetes, age related macular degeneration, endometriosis, unwanted corneal angiogenesis, and the like. Any condition which would be benefited by inhibiting angiogenesis and/or destroying neovasculature selectively, would be benefited by the invention method.

The acute phase of the treatment employs administration of targeted antiproliferative or antiangiogenic agents. The targeting agents employed are those that characterize neovasculature or the location at which angiogenesis is taking place. Neovasculature in general can be targeted by targeting integrins such as αᵥ↑₃, αᵥβ₅, and α₅β₁ by targeting receptors for angiogenic kinases such as the VEGF receptor, selectins, such as e-selectin and p-selectin, moieties that target adhesion molecules such as VCAM, and, in some cases, utilizing delivery vehicles that will be entrapped in dysmorphisms characteristic of neovasculature. In some conditions, the location of the neovasculature will offer an environment that can itself be targeted. For example, for treatment of tumors, antibodies or other agents that target tumor-specific epitopes may be used. In the case of atherosclerosis, it may be useful to target the vasa vasorum.

Blood vessels typically comprise a hollow lumen surrounded by a median, which is in turn surrounded by an adventitial layer comprising the vasa vasorum. In atherosclerosis plaques, angiogenic vessels primarily develop from the vasa vasorum in the adventitial layer of the plaque and extend into the thickening intimal layer of the atheroma. They generally do not originate from the primary arterial lumen. Neovasculature proliferation has been localized to atherosclerotic plaque and in particular to lesions clinically associated with unstable angina, myocardial infarction and stroke. Plaque angiogenesis plays a role in promotion of plaque growth, intraplaque hemorrhage, and lesion instability.

An antiangiogenic or antiproliferative agent refers to an agent that enhances the growth of blood vessels. These terms are sometimes used interchangeably herein; however, a required effect is encouraging the proliferation that results in neovasculature.

Targeting agents for neovasculature in the vasa vasorum may target, for example, αᵥβ₃ integrin, α₅β₁, αᵥβ₅ or the receptor for VEGF. A wide variety of targeting agents can be employed including antibodies directed against these targets, and various peptidomimetics as described, for example, in U.S. Pat. Nos. 6,322,770; 6,130,231; 6,153,628 and PCT publication WO 01/97848. Also useful are aptamers, specific endothelial cell targeting proteins such as TAT (derived from HIV), or candidates screened from libraries of small molecules. Methods for coupling such agents to fluorocarbon nanoparticles are described in PCT publication WO 2003/062198.

In the acute phase of the invention protocols, the antiproliferative or antiangiogenic agent is targeted, as set forth above, to the neovasculature itself or to the location at which angiogenesis occurs. Any method of associating the targeting agent with the antiproliferative agent may be used, including simple linkage or use of bifunctional antibodies. In many cases, however, it is convenient to associate both the targeting agent and the antiproliferative agent with particulate delivery vehicles. Many suitable types of delivery vehicles are known in the art and could be employed.

These include liposomes, micelles, polymeric matrices, fluorocarbon based particles, solid, liquid, or gas phase particles, and the like. The size of the particulate delivery vehicles should be suitable for parenteral administration. The administration is typically intravenous, although other routes of parenteral administration may also be used. Other particulates include oil in water emulsions and emulsions of halogenated hydrocarbons. Suitable alternative carriers are described in PCT publication WO 2005/077407 referenced above.

Similarly, the '407 PCT publication describes drugs that are useful as acute antiangiogenic drugs. The list of drugs disclosed in this publication is incorporated herein by reference. Included among these antiangiogenic drugs that may be administered for acute treatment are matrix metalloprotease inhibitors, protein kinase C-β inhibitors, vascular endothelial growth factor (VEGF) inhibitors, basic FGF binding molecules, paclitaxel, rapamycin, fumagillin, doxorubicin and many others. Any antiangiogenic drug may be used in the acute phase of the invention.

Of course, combinations of antiproliferative/antiangiogenic agents may be employed in the acute treatment and the acute phase may include administration of other beneficial drugs along with the at least one antiproliferative/antiangiogenic agent.

In the acute phase, targeted antiproliferative/antiangiogenic agents are administered for a limited time and then administration is stopped over at least part of the evaluation period. Additional acute treatments may also occur at later points during the evaluation period. For example, if the evaluation period is 6 months, acute treatment might take place every month or every two months.

The method of the invention does not require actual evaluation by any particular means, but this is not precluded, and can be conducted periodically according to the invention method. If the practitioner desires to employ evaluation techniques to monitor the effectiveness of the combined treatment, the location and level of angiogenesis can be monitored by a variety of means. Any appropriate imaging technique can be used. In one embodiment, targeted suspensions of nanoparticles comprises MRI contrast agents enhance the quality of the image of the angiogenic sites in the subject. Images may be obtained at various timepoints during the evaluation period to assess the progress of the treatment.

While no particular evaluation method is required, the present inventors have found that the method employed in the examples below is particularly successful. By having a straightforward method to evaluate the status of neovasculature and angiogenesis, it is possible to modify the treatment, including the balance between acute and chronic phases, in order to modify the treatments as necessary. For example, if it appears that although angiogenesis was initially inhibited by acute administration, growth of neovasculature has again resumed, a further administration of acute agent might be indicated. If, on the other hand, there is no relapse in terms of neovasculature formation, further acute treatment may not be necessary and chronic treatment alone may be sufficient. The ability to monitor the progress of neovasculature by repeated administration of targeted nanoparticles, in particular nanoparticles that consist of perfluorocarbon cores coated with lipid/surfactant which are targeted to markers for neovasculature such as integrins, and which bear contrast agents such as chelated paramagnetic metal ions greatly improves the efficacy and nuances of the treatment regimen.

By “periodic” evaluation, is simply meant evaluation at more than one point during the evaluation interval or evaluation period. Typically, the evaluation will be repeated 3-10 times or more during the evaluation period and preferably at regular intervals.

Turning, now, to the chronic aspect of the treatment, this is continuous throughout at least 50%, or 70% or 80% over the entire evaluation period.

As noted above, the regimen for the chronic administration is preferably one that can conveniently be self-administered. Typically, such chronic administration employs dosing once or twice or three times per day or once every two days or once every three days. The chronic administration may be repetitive rather than continuous, and indeed typically is.

The agents that are administered on a chronic basis may be of considerable variety and are designed to complement the acute treatment so as to prolong the effect of the acute treatment. In one important embodiment, the agent administered on a chronic basis will include drugs that are themselves antiangiogenic or antiatherosclerotic.

The primary or most readily recognized biological activity of the chronically administered therapeutic agent may not be antiproliferation/antiangiogenesis. However, the drug must include this as one of its properties or side effects. Indeed, it would be possible to use the same drug for both chronic and acute administration. Alternatively, a drug that was primarily antiproliferative/antiangiogenic could be used in the chronic segment of the protocol. Thus, if paclitaxel, rapamycin, fumagillin or doxorubicin is used in the acute treatment, it would also be possible to use one or another of these in the chronic phase. Because many drugs have antiangiogenic side effects, a wide range of drugs, however, may be used.

One class of drugs that is commonly employed to help people avoid atherosclerosis is the statins due to their ability to control cholesterol levels. These are also useful as chronic treatment in the invention protocols, in view of their ability to inhibit angiogenesis, as an additional property.

Thus, typical drugs that are administered on a chronic basis include the statins, such as atorvastatin, simvastatin, lovastatin, pravastatin, mevastatin, and the like. Typically, the chronic drug is administered orally, although parenteral administration may also be used.

Other drugs for chronic treatment include antioxidants such as coenzyme Q, certain B vitamins such as folic acid, B6 and B12 which reduce homocysteine levels, nicotinic acid treatment, fibric acid derivatives such as clofibrate, gemfibrozil, and fenofibrate, cholesterol transport blockers such as probucol, and non-absorbable resins such as cholestyramine and cholestipol. All of these drugs for chronic protocols are administered as part of a specified protocol that involves the acute administration of agent as well and does not include chronic administration often practiced by individuals on their own, such as taking One-A-Day® vitamins or iron supplements. The chronically administered drug must, in any event, be antiangiogenic at some level. The specific antiangiogenesis drugs used in the acute protocol can also be used in the chronic regimen.

The protocols by which the drugs may be administered are varied, but typically, the antiangiogenic drugs suitable for acute treatment in targeted nanoparticles or other delivery vehicles will be administered over a short time period such as hours or days often in repetitive dosages spaced by intervals suitable to the subject. The antiangiogenic drug (or drugs) or other drugs as listed above is (are) administered in a chronic regimen over longer time periods, either after, before, during or typically during and after the administration of the “acute” antiangiogenic drug.

When it is desired to monitor the progress of treatment, any appropriate method may be used, but a convenient method utilizes targeted MRI agents as described hereinabove. In the case of atherosclerotic plaque, an enhancement of contrast over time indicates increased plaque, whereas decreased contrast over time indicates that the plaque is being successfully treated.

By “treating” is meant improving the condition of the subject. Thus, the neovasculature may simply be arrested and stabilized, may be prevented in subjects at risk, or may be destroyed or diminished. For example, in the case of atherosclerosis, treatment may prevent the formation of plaques prior to their detection, in particular in subjects at high risk for plaque formation, may decrease plaque size, may arrest of plaque growth, or any other amelioration of what would otherwise be the negative condition of the subject with respect to plaque formation and maintenance.

The subjects amenable to treatment are typically humans, but other warm-blooded animals such as livestock (e.g., sheep, cattle, pigs, goats) and companion animals (e.g., cats, dogs) can also be successfully treated by the methods of the invention. In addition, animal models for the various conditions identified by the invention method for atherosclerosis treatment such as mice, rats and rabbits are useful to optimize the appropriate combination of acute and chronic treatment.

### EXAMPLES

The following examples are intended to illustrate but not to limit the invention.

### EXAMPLE 1

### Effect of Combined Treatment

Cholesterol-fed rabbits received αᵥβ₃-targeted paramagnetic nanoparticles (NP) with (n=9) or without (n=9) fumagillin (30 μg/kg body weight) at weeks 0 and 4. A portion of the animals (n=4 per group) was treated with atorvastatin included in the high-cholesterol chow (44 mg/kg feed). Assessment of antiangiogenic response was evaluated at weeks 2, 4, 6 and 8 using αᵥβ₃-targeted paramagnetic NP lacking the drug. MRI signal enhancement from the aortic vasa vasorum neovasculature was calculated from transverse black-blood MR images (1.5 T) collected before and 4 hours post NP injection (1 ml/kg body weight). The MRI enhancement observed at each timepoint was normalized with respect to the enhancement measured at week 0.

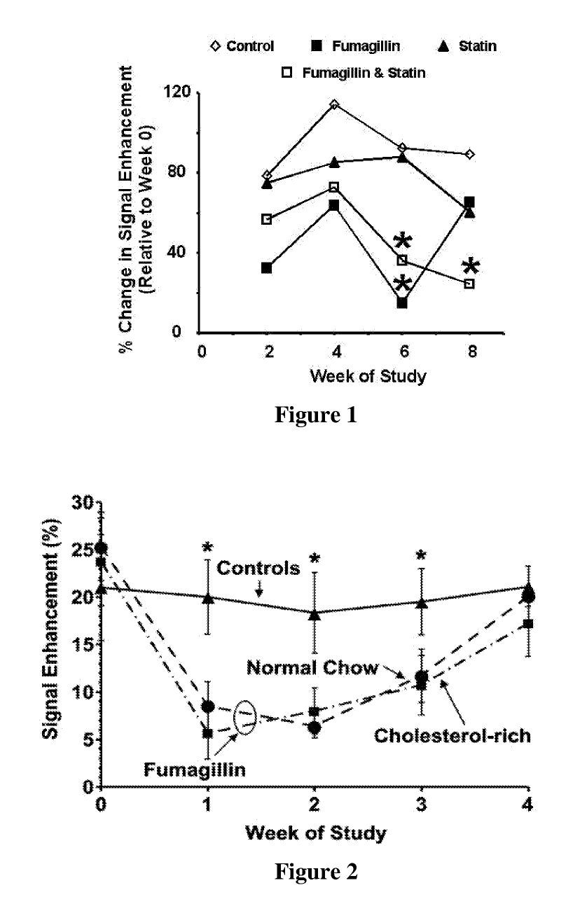

The results are shown in FIG. 1. At all timepoints, MRI enhancement in the thoracic aorta of control rabbits was similar to the value observed at week 0. Enhancement in rabbits receiving αᵥβ₃-targeted fumagillin NP, with or without statin, was lower at week 2 compared with the baseline values. Rabbits receiving only αᵥβ₃-targeted fumagillin NP had decreased enhancement at week 6, which increased at week 8 reflecting recurrence of pathological angiogenesis 4 weeks after the last NP treatment. Treatment with only atorvastatin showed a slow decrease in enhancement reflecting the gradual antiangiogenic effect of oral statins.

Rabbits receiving αᵥβ₃-targeted fumagillin NP and atorvastatin showed reduced enhancement at weeks 6 and 8 (* p<0.05), indicating sustained suppression of neovascular proliferation despite the intervening weeks since the last NP injection.

### EXAMPLE 2

In this example, the effect of chronic administration of statins in combination with targeted acute administration of fumagillin was compared to the combination of fumagillin administration with a cholesterol rich or normal cholesterol diet. The results confirm that the amount of cholesterol in the diet had no detectable effect.

The techniques employed were those described by Winter, P. M., et al., Arterioscler. Thromb. Vasc. Biol. (2006) 26:2103-2109 cited above, modified as described below.

In a first experiment, male New Zealand white rabbits were fed a 0.5% cholesterol diet for 80 days whereas a control group was fed a normal non-hyperlipidemic diet for a comparable period. On day 0, targeted fumagillin coupled to nanoparticles as described in Winter (supra) was administered to rabbits wherein the nanoparticles contained 0.2 mole % fumagillin or 0.6 mole % fumagillin, again, as calculated in the Winter paper. A control group was administered similar particles that did not contain fumagillin. Each test group contained members with the high cholesterol diet and regular chow.

The groups were followed over a period of four weeks and images were obtained weekly using targeted paramagnetic nanoparticles that did not contain drug, again as described by Winter. MRI signal enhancement from the aortic wall was averaged over all image slices using semi-automated segmentation programs as described previously.

The results are shown in FIG. 2. As shown, controls maintained essentially the same signal enhancement indicating angiogenesis over the four week period. The rabbits administered 0.2 mole % particles comprising fumagillin showed dramatic declines in signal enhancement after one week, which were maintained until about week 3. At week 4, the signal enhancement had returned essentially to its original level. The same pattern was obtained whether the rabbits were fed normal chow or a cholesterol-rich diet.

It was also found that the results did not vary if the level of fumagillin was increased to 0.6 mole % (results not shown).

### EXAMPLE 3

### Effect of Chronic Statin Administration

In this example, all of the rabbits were fed a 0.5% high cholesterol diet over 80 days and were divided into five groups as follows:

(1) αᵥβ₃-targeted nanoparticles no atorvastatin

(2) αᵥβ₃-targeted nanoparticles with atorvastatin (1.75 mg/kg/day in all groups receiving atorvastatin)

(3) αᵥβ₃-targeted fumagillin (0.2 mole %) nanoparticles at week 0+atorvastatin

(4) αᵥβ₃-targeted fumagillin (0.2 mole %) nanoparticles on weeks 0 & 4 with no atorvastatin

(5) αᵥβ₃-targeted fumagillin (0.2 mole %) nanoparticles on weeks 0 & 4 with atorvastatin

Rabbits were imaged at 1 week, 2 weeks, 4 weeks, 6 weeks and 8 weeks using αᵥβ₃-targeted paramagnetic nanoparticles (no drug). MRI signal enhancement from the aortic wall was averaged over all imaged slices using a custom, semi-automated segmentation program as before.

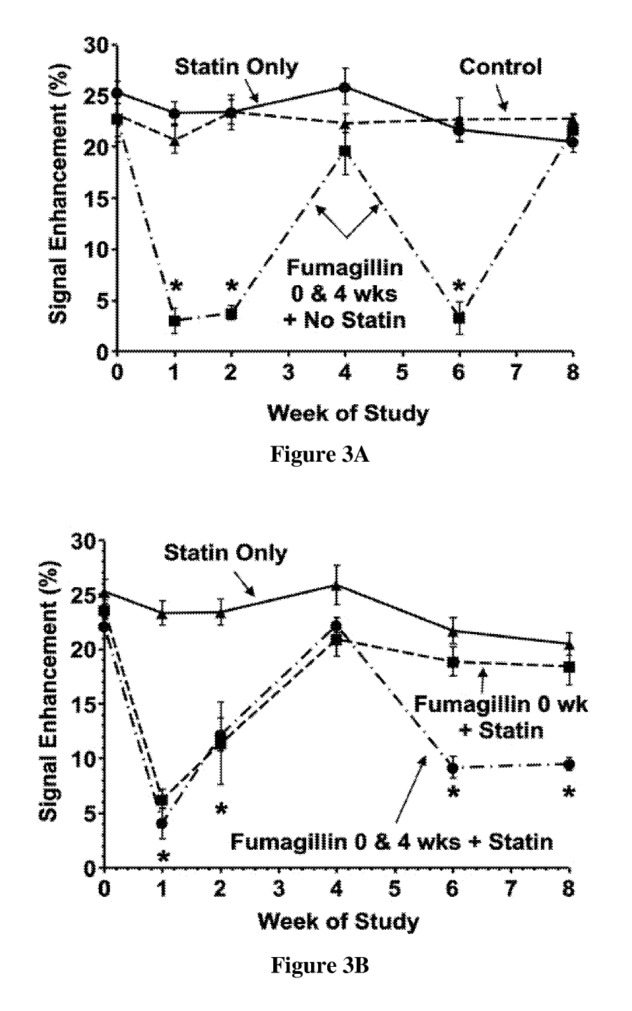

The results are shown in FIGS. 3A and 3B. As shown in FIG. 3A, which compares the signal enhancement in rabbits either provided the control particles and no atorvastatin (group 1); atorvastatin only (group 2) with the group administered fumagillin at 0 and 4 weeks without atorvastatin (group 4). As seen in FIG. 3A, providing targeted fumagillin dramatically lowered the signal enhancement after 1 week, which was maintained until week 2, but at week 4 rebounded to its original level. The additional fumagillin administration at week 4 then again reduced the signal essentially in the same pattern as the week 0 treatment—decreasing dramatically at a two-week timepoint but then returning to its higher level.

FIG. 3B shows a comparison of the group administered only atorvastatin (group 2) with groups that were administered fumagillin only at the beginning on the experiment, along with atorvastatin (group 3) and the rabbits administered fumagillin at 0 and 4 weeks along with atorvastatin (group 5). From the results in FIG. 3B, it is apparent that the continued administration of statin after fumagillin was only administered at week 0 was unable to prevent return to higher values of signal enhancement. However, after the second injection of fumagillin at week 4 in the presence of atorvastatin the lowered signal enhancement was maintained over a 4-week period, in contrast to similar protocol without atorvastatin. A comparison of this result in FIG. 3B with the result for the group with 0 and 4 weeks fumagillin administration but no statin in FIG. 3A shows that in this regimen, statin is able to maintain the results of fumagillin after the second administration.

1. A method to treat a condition in a subject characterized by unwanted neovasculature, which method is a protocol that comprises:
acutely administering to a subject in need of such treatment an effective amount of an antiangiogenic composition comprising at least one antiangiogenic agent targeted to the neovasculature or to the location of neovasculature; and
chronically administering at least one additional therapeutic agent over time.

2. The method of claim 1, wherein said antiangiogenic composition comprises particulate delivery vehicles comprising said antiangiogenic agent, wherein said delivery vehicles are targeted to the neovasculature.

3. The method of claim 2, wherein the delivery vehicles are targeted to an integrin.

4. The method of claim 1, wherein acutely administering antiangiogenic composition is by parenteral administration and administering the additional therapeutic agent is by oral administration.

5. The method of claim 1, wherein chronic administering of the additional therapeutic agent is performed during and after acutely administering the antiangiogenic composition.

6. The method of claim 2, wherein the delivery vehicles are fluorocarbon nanoparticles coated with a lipid/surfactant layer.

7. The method of claim 1, wherein the antiangiogenic agent is fumagillin, rapamycin, and/or a taxane, and/or
wherein the additional therapeutic agent is a statin.

8. The method of claim 1, wherein the subject is human, or is an animal model for a human condition.

9. The method of claim 1, wherein the condition is atherosclerosis, cancer, arthritis, or an eye related condition.

10. The method of claim 9, wherein the condition is atherosclerosis.

11. The method of claim 1, which further includes periodically evaluating the effectiveness of the protocol at least during the time of chronically administering.

12. The method of claim 11, wherein said periodic evaluation employs MR imaging of neovasculature.

13. The method of claim 12, wherein said MR imaging employs targeted MRI contrast agent.

14. The method of claim 13, wherein the targeted MRI contrast agent consists essentially of chelated paramagnetic ions coupled to nanoparticles targeted to said neovasculature.
